(19) United States
(12) Patent Application Publication
(10) Pub. No.: US 2011/0237500 A1
(43) Pub. Date: Sep. 29, 2011

(54) CECROPIN-MAGAININ HYBRID PEPTIDES

(75) Inventors: Marc Alan Fox, Salisbury (GB); Joanne Elizabeth Thwaite, Salisbury (GB); Timothy Philip Atkins, Salisbury (GB)

(73) Assignee: The Secretary of State of Defence, Salisbury, Wiltshire (GB)

(21) Appl. No.: 13/131,829

(22) PCT Filed: Nov. 30, 2009
(86) PCT No.: PCT/GB2009/002784

§ 371 Date: May 27, 2011

(30) Foreign Application Priority Data
Nov. 28, 2008 (GB) 0821707.7

(51) Int. Cl. A61K 38/17; C07K 19/00; C07H 21/04; A61P 31/04; A61P 31/12; A61P 31/10

(52) U.S. Cl. 514/2.3; 530/326; 536/23.4

(57) ABSTRACT

The present invention relates to a biocidal fusion peptide of SEQ ID NO: 1 or a sequence with at least 90% identity thereto, compositions such as pharmaceutical compositions comprising the same, methods of preparing the peptide, and use of the peptide in treatment, in particular for the treatment of bacterial infection and/or fungal infection and/or viral infection.

The present disclosure relates to fusion peptides, for example with antibacterial and/or anti-fungal and/or anti-viral activity, composition such as pharmaceutical compositions comprising the same, methods of preparing the peptides, and use of the peptides in treatment, in particular for the treatment of bacterial infection and/or fungal infection and/or viral infection.

Many different families of anti-microbial peptides, classified by their amino acid sequence and secondary structure have been isolated from insects, plants, mammals and microorganisms.

Melittin is a peptide with antibacterial activity isolated from honey bee venom.

Cecropin, cysteine-containing defensin and sapecin, isolated from insects, are examples of antibacterial peptides whose target site is lipid membrane of Gram positive bacteria. Studies have demonstrated that Cecropin B isolated from Bombix mori has biological activity against bacterial species. Further, it was reported that this peptide when translocated into the intercellular spaces in rice transgenic plants was protected from degradation by plant peptidases and confers enhanced resistance of the rice plants against Xanthomonas oryzae pv. oryzae infection.

Attacin, sarcotoxin, deftericin, coleoptericin, apidaecin and abaecin are other antibacterial peptides whose target site is lipid membranes. These peptides conserve G and P domains, and have an influence on the cell differentiation of Gram negative bacteria. In particular, attacin has been also reported to break down outer membrane of the targeted bacteria by inhibiting the synthesis of outer membrane proteins.

Sarcotoxin IA is an antibacterial peptide that is secreted by a meat-fly Sarcophaga peregrina larva in response to a hypodermic injury or bacterial infection. This peptide is highly toxic against a broad spectrum of both Gram-positive and Gram-negative bacteria and lethal to microbes even at nanomolar concentrations.

Several antibiotic peptides have been also isolated from amphibia, and many of them belong to the group of amphipathic alpha-helical structure peptides such as magainins, bombinins, bufonins, dermaseptins and defensins.

According to Zasloff (1987), at least five proteins may be isolated from the skin of the African clawed frog (Xenopus laevis). The natural proteins are active against a broad range of microorganisms including bacteria, fungi and protozoans.

WO 03/010191 describes an anti-microbial peptide, isolated from skin of Phyllomedusa hypochondrialis, a kind of frog native to Amazonian, Brazil.

Anti-microbial activity was described in the U.S. Pat. No. 5,643,876 for peptides derived from magainin. These peptides have a molecular weight of about 2500 Da or less, are highly water soluble, amphiphilic and non-hemolytic.

U.S. Pat. No. 5,424,395 discloses a synthetic peptide with a 23 amino acid sequence, derived from magainin II showing anti-microbial activity in plants. U.S. Pat. No. 5,912,231 discloses a compound comprising a magainin I or a magainin II peptide with biological activity, wherein at least one amino acid residue may be substituted with other amino acids residues.

Fusions polypeptides of cecropin and melittin were prepared in Bhargava et al Biophys. J. 2004 January 86(1); 329-336 for investigating the mechanisms employed by the peptides.

WO 2006/138276 also described cecropin and melittin hybrids such as CEME and CEMA, with reduced toxicity to host transgenic plants.

WO 90/11771 discloses antibiotic hybrid peptides where the components are selected from cecropin, cecropin A, cecropin B, cecropin D, melittin, magainin or attacin, with anti-malarial and/or anti-bacterial activity.

Whilst a number of peptides are known they have not been developed for use as antimicrobial agents because generally there are one or more disadvantages associated with their activity, for example some are toxic and have haemolytic effects, and others simply do not have the broad spectrum activity or sufficiently strong activity to render them therapeutically useful.

The present disclosure relates to peptides with advantageous and/or optimised properties and provides a biocidal fusion peptide of Sequence ID No: 1 or a sequence with at least 90% identity thereto.

In one embodiment the peptide of the disclosure is predicted to have or adopt an alpha-helix structure under appropriate conditions.

The use of magainin such as a magainin II seems to provide advantageous properties on the fusion peptides prepared. Nevertheless there seems to be a large amount of unpredictability with the activity of peptides employing magainin for example a fusion peptide of cecropin 1-8 and magainin 1-12 does not have comparable activity to the peptide of the currently claimed disclosure. Given the high levels of unpredictability in relation to the activity of peptides comprising magainin it is surprising the peptide disclosed herein has optimized properties in that it has broader activity than at least magainin but alternatively or additionally cecropin. Furthermore, the fusion peptide seems to more active than magainin and/or cecropin.

Thus the fusion peptides of the disclosure may be particularly advantageous in the variety of organisms/pathogens against which they are active. In addition to the advantageous anti-bacterial, anti-fungal and/or anti-viral profile the peptides of the disclosure may also have lower toxicity, than certain known antibacterial peptides thereby making them more suitable for administration to humans and/or animals.

The peptides according to the disclosure are suitable for treatment of humans and/or animals and at the doses administered/employed they are not cytotoxic to cells.

In one embodiment the peptide of the disclosure peptide is 91, 92, 93, 94, 95, 96, 97, 98 or 99% homologous/identical to a peptide of Sequence ID No: 1 when analysis is performed against the full length of the sequences being compared.

Analysis for sequence identity/homology may be performed employing software such as BLAST. Degrees of identity can be readily calculated using known computer programs (see Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). For example, simple sequence comparisons can be done on web-sites such as the NCBI website: http://www.ncbi.nlm.nih.gov/BLAST/ (version 2.2.11). As used herein, percentages identity between sequences are measured according to the default BLAST parameters, version 2.2.11. For polypeptides, blastp is used, for example, with the following settings: advanced blasting, low complexity, expect 10, word size 3, blosun 62 matrix, existence: 11, extension: 1 gap costs, inclusion threshold 0.005 and alignment view: hit table. For nucleotide blasting, blastn is used, with low complexity, expect 10, wordsize 11, alignment view: hitable, semi-auto and autoformat.

Biocidal in the context of the context of the present disclosure is intended to refer to peptides capable of damaging, killing, destroying or neutralising pathogens, in particular micro-organisms (such as bacteria) and/or viruses. Pathogen in the context of the present application is not intended to refer to parasites such as malarial parasites.

In one or more embodiments the peptides of the disclosure are bioactive prokaryotic cell-membrane interfering peptides.

Bioactive prokaryotic cell-membrane interfering peptides are those which are capable of infiltrating, disrupting, pore forming, thereby by damaging or destroying the membrane of a prokaryotic cell.

In one embodiment the peptide of the disclosure is comprised within a larger peptide entity, provided the sequence of Sequence ID No: 1 or a sequence at least 90% identical thereto is present as a contiguous amino acid sequence.

The targeting of the micro-organisms may be optimized by an overall, net charge on the peptide, such as a positive or negative charge, in particular positive charge. It may be that the net positive charge may assist the polypeptide targeting the bacteria, which have a net negative charge. In contrast human and animal cells are neutral and thus in this scenario would be less likely to interact with the net charged peptide, thereby making it more likely that the peptide would interact with an oppositely charged microbe in the vicinity.

The biocidal peptides of the disclosure are predicted to have an alpha-helix type structure with a hydrophobic loop capable of targeting the lipophilic layer in the target membrane. This structure is thought to be important to the activity of the peptides, herein.

Most of the peptides without disulfide bridges have random structures in water, and when they bind to a membrane or other hydrophobic environment, or self-aggregate, they form an alpha-helical structure. For example, cecropins and melittin only acquire amphiphilic alpha-helices in membranous environments. It is known that the both dual cationic and hydrophobic nature of the peptides is important for the initial interaction between the peptide and the bacterial membrane.

Software programmes such as SIMPA ('96) can be employed to predict the structure of the peptide from the amino acid structure, thereby allowing optimized structures to be prepared.

It may be advantageous to group hydrophilic residues such that when a helix is formed they are grouped together to form a hydrophilic region. This may reduce the positivity angle of the peptide/helix and ensure efficient docking of the peptide with the target cell membrane. The 2D structure of the helix represented from above may, for example, be referred to by a model known as a Schiffer-Edmonson wheel structure. See for example Biophysical Journal 1967 Vol. 7, page 121 to 135. The structure of the wheel can be used to predict the amino acid grouping, which will be encountered by the target cell membrane. Amino acids may be omitted or substituted to achieve, for example a hydrophilic or hydrophobic section in a part of the molecule, depending on exactly what is required.

In one embodiment the positivity angle is less than 150 degrees.

In one embodiment the positivity angle is about 100 degrees or less.

Whilst not wishing to be bound by theory it is thought that the specific peptides disclosed herein adopt an alpha-helix structure, at least, when in an appropriate membrane environment.

The bioactive heterologous peptides which can be employed in peptide (or other entity) comprising the amino acid sequence of Sequence ID No 1 or a sequence at least 90% identical thereto (including active fragments/active domains therefrom) are those which are able to adversely affect the normal function of the a microbial or viral cell for example causing disruptions of the cell membrane, lysis, death prevention of proliferation and/or prevention growth/mitosis or the like. Whilst the original heterologous peptide should have the latter activity unfused fragments of the same may not necessarily have the activity, until incorporated into a fusion peptide. The bioactive heterologous peptides may, for example be selected from Buforin I, buforin II, bactolysins, attacin, sarcotoxin, deftericin, coleoptericin, apidaecin and abaecin, cecropin, defensin, sapecin, and/or dermaseptins, melittin, magainins (such as magainin II), derived from Xenopus laevis, or derived from Phyllomedusa hypochondrialis and/or LL-37.

The sequence of wild-type Magainin II is GIGKFLHSAKKFGKAFVGEIMNS [Sequence ID No: 2].

An alaninated derivative with the S⁸, G¹³ and G¹⁸ residues all replaced with As (GIGKFLHAAKKFAKAFVAEIMNS [Sequence ID No: 3]) has also been reported by Chen et al (1988) FEBS 236 (2), 462-466.

Derivatives of magainin II include for example:

(SEQ ID NO: 4)
Gly Ile Gly Lys Phe Leu His Ser Ala Lys Lys Phe
Gly Lys Ala Phe Val Gly Ile Met Lys Ser;
(SEQ ID NO: 5)
Gly Ile Gly Lys Phe Leu His Ser Ala Lys Lys Phe
Gly Lys Ala Phe Val Ala Ile Met Lys Ser;
(SEQ ID NO: 6)
Gly Ile Gly Lys Phe Leu His Ser Ala Lys Lys Phe
Gly Lys Ala Phe Val Phe Ile Met Asn Ser*,;
wherein Ser* is D-Serine
(SEQ ID NO: 7)
Gly Ile Gly Lys Phe Leu His Ser Ala Lys Lys Phe
Phe Lys Ala Phe Val Phe Ile Met Asn Ser;
(SEQ ID NO: 8)
Gly Ile Gly Lys Phe Leu Lys Ser Ala Lys Lys Phe
Gly Lys Ala Phe Val Phe Ile Met Asn Ser;
(SEQ ID NO: 9)
Gly Ile Gly Lys Phe Leu His Lys Ala Lys Lys Phe
Ala Lys Ala Phe Val Phe Ile Met Asn Ser;
(SEQ ID NO: 10)
Gly Ile Gly Lys Phe Leu Lys Ser Ala Lys Lys Phe
Ala Lys Ala Phe Val Phe Ile Met Asn Ser;
(SEQ ID NO: 11)
Gly Ile Gly Lys Phe Leu His Lys Ala Lys Lys Phe
Ala Lys Ala Phe Val Phe Ile Met Asn Lys;
(SEQ ID NO: 12)
Gly Ile Gly Lys Phe Leu Lys Lys Ala Lys Lys Phe
Gly Lys Ala Phe Val Phe Ile Met Lys Lys;
and
(SEQ ID NO: 13)
Gly Ile Gly Lys Phe Leu His Ser Ala Lys Lys Phe
Gly Lys Ala Phe Val Xaa Ile Met Asn Ser;.
wherein Xaa is .epsilon.-Fmoc-lysine

In one embodiment the peptide is formed such that domains from one peptide form a sandwich around the sequence of the present disclosure.

In another embodiment the sequences from a further heterologous peptide are inserted at the beginning, the N-terminal, or the end, the C-terminal, of the peptide of the present disclosure.

Fusion peptide in the context of the present disclosure may include a molecule with 50 amino acids or less, for example 40 or less such as about 10 to 37, particularly 17 to 27, such as 21 to 25.

In one aspect the fusion peptide employed has a weight of 50,000 Daltons or less, such as 40,000, 30,000, or 25,000 Daltons.

The peptides of the disclosure may be effective antibacterial agents, antiviral agents and/or antifungal agents.

Given that the peptides of the disclosure attack the cell membrane of the target entity it is difficult for the target microbes to mutate and become resistant to the peptide. This is important because bacteria have been able to mutate to become resistant to many known types of antibiotics.

In another embodiment up to one amino acid in 10 in the sequence is replaced/substituted with an alternative amino acid provided the biological function of the sequence is retained. In one embodiment the replacements are conservative replacements.

In one embodiment a total of 1 to 10, such as 1 to 5, in particular 2, 3 or 4 amino acids are substituted or deleted in comparison to the relevant portion of the original peptide.

The disclosure also extends to pharmaceutical composition comprising a fusion peptide as defined herein, and a pharmaceutically acceptable excipient such as a diluent or carrier.

In one embodiment the formulation is for topical administration to a wound in the derma or for administration to the lungs. In this embodiment the formulation may be provided as a solution wherein the diluent is, for example saline, sterile water, a dextrose solution or phosphate buffer solution. Liquid formulations may also contain other ingredients for example preservatives, such as benzalkonium chloride, which is commonly used in pharmaceutical compositions.

Formulations for topical administration, including for administration to the lungs, may also be formulated as dry powders, for inhalation or for dusting the wound or infected area. Dry powder formulations may comprise, for example lactose and will need to have a particle size less than 10 microns if the formulations are to be administered to the lungs. Dry powder formulation may be particularly useful in the treatment of fungal infections, such as athlete's foot, onychomycosis, tinea unguium, pityriasis versicolor and/or candida albicans.

Suitable formulations wherein the carrier is a liquid (including a solution or suspension) for administration as, for example, nasal spray, nasal drops, or by aerosol administration by nebulizer, may include aqueous or oily solutions of the active ingredient.

Liposome carriers when employed in the formulations of the disclosure may serve to target a particular tissue or infected cells, as well as increase the half-life of the active. Liposomes include emulsions, foams, micelles, insoluble monolayers, liquid crystals, phospholipid dispersions, lamellar layers and the like. Liposomes may be formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka, et al., Ann. Rev. Biophys. Bioeng. 9:467 (1980), U.S. Pat. Nos. 4,235,871, 4,501,728, 4,837,028, and 5,019,369.

The liposomes generally contain a neutral lipid, for example phosphatidylcholine, which is usually non-crystalline at room temperature, for example eggyolk phosphatidylcholine, dioleoyl phosphatidylcholine or dilauryl phosphatidylcholine.

In one embodiment the formulation is provided as a formulation for infusion. The peptide may for example be lyophilised for reconstitution with sterile water or aqueous buffer solution.

The disclosure also extends to use of peptides or compositions comprising the same as defined herein for the treatment or prophylaxis of bacterial and/or viral infections and/or fungal infections (for example as described herein).

The fusion peptides of the disclosure may be particularly useful in the treatment of S aureus and/or B. cepacia and/or Y. pseudotuberculosis infections.

The peptides according to the disclosure also appear to be particularly useful for neutralising B. anthracis spores and/or B. anthracis vegetative and/or B. subtilis spores and/or B. subtilis vegetative.

The disclosure also includes methods of treatment or prophylaxis of bacterial and/or viral infections and/or fungal infections comprising administering a therapeutically effective amount of a peptide or composition as described herein.

Whilst the dose will depend on a number of variables such as the age and weight of the patient and infection being treated, a dose in the range 1 μg to 500 mg per Kg may be suitable, for example 10 μg to 1 mg per Kg.

When administered topically to the skin as a solution formulation a concentration in the range 0.1 to 10% w/w or w/v such as 0.5 to 5% w/w or w/v may be appropriate.

Also encompassed within the scope of the present disclosure is a detergent formulation as an antibacterial and/or antiviral agent and/or antifungal agent for treating surfaces (comprising a peptide as defined herein).

The disclosure also relates to use of peptides herein as preservatives, for example in food preparation, cosmetics and/or pharmaceutical formulations and products.

The disclosure also relates to polynucleotide sequences such as DNA sequences encoding said peptides.

The disclosure also extends to hosts comprising said encoding polynucleotides.

A method of preparing a peptide recombinantly in a host is also provided.

It is also envisaged that one or more embodiment described herein may be combined, as technically appropriate. In the context of this specification “comprising” is to be interpreted as “including”. Aspects of the disclosure comprising certain elements are also intended to extend to alternative embodiments “consisting” or “consisting essentially” of the relevant elements.

### EXAMPLES

Strains were grown to mid-exponential phase in LB broth at 37° C. or used in their sporulated form. Aliquots of these cultures containing approximately 1×10⁶ CFU/ml were separately exposed to PBS (control) or 102 μg/ml of each peptide. Cultures were maintained at 37° C., 180 rpm throughout the assay. Samples were taken at 0, ½, 1, 2, 3 and 4 hours, then serially diluted in PBS and enumerated on LB agar. Viable CFU/ml counts were obtained following incubation at 37° C.

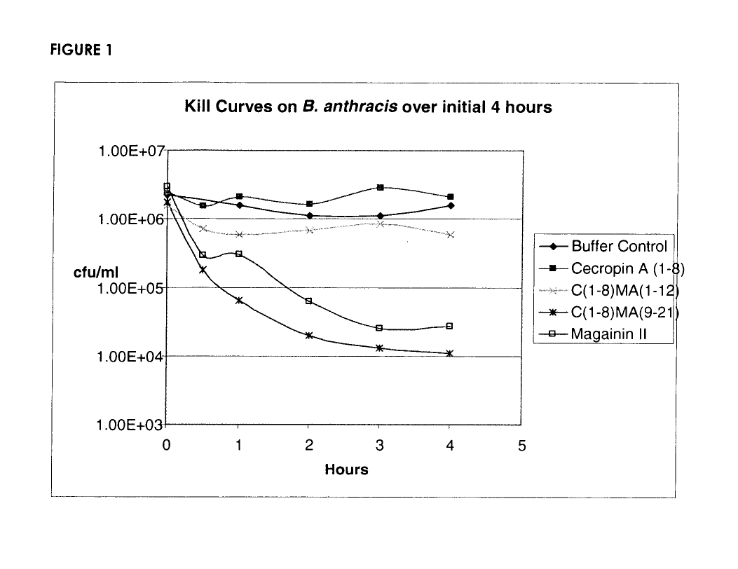

The results for spores are shown in FIG. 1, which indicates that the fusion peptide C1-8MA1-12, cecropin fragment 1-8 and buffer had no antimicrobial activity in the assay performed.

Table 1 below indicates that the peptide of Sequence ID No: 1 was active against F. tularensis in addition to the pathogens against which magainin II was active against. In addition the peptide of the disclosure had greater activity and/or more rapid activity against B. anthracis Spores, B. cepacia, and S. aureus than magainin II.

TABLE 1
Effect of peptides after 4 hours incubation with bacteria

| | B. anthracis | B. anthracis | | B. | | Y. pseudo- | | | |
|---|---|---|---|---|---|---|---|---|---|
| | Spores | Vegetative | B. cepacia | thailandensis | Y. pseudotuberculosis | tuberculosis | F. novicida | F. tularensis | S. aureus |
| | UM23-Cl2 | UM23-Cl2 | J2540 | E264 | IP32593 | YPIII | U112 | LVS | ATCC29213 |
| Magainin II | ** | *** | ** | — | — | *** | — | — | **** |
| CaMA | *** | *** | “***” | — | — | “****” | — | *** | “****” |

Key
— No effect (i.e. no significant difference between control [culture with no peptide] and culture with peptide)
* ~1 log effect (i.e. culture with peptide that's growth was ~1 log less than control)
** 1-2 log effect
*** 2-4 log effect
**** >4 log effect
“Immediate effect” (i.e. loss of growth by at least 1 log compared to control immediately on addition of peptide [T = 0])

1. A biocidal fusion peptide of Sequence ID No: 1 or a sequence with at least 90% identity thereto.

2. A pharmaceutical composition comprising a fusion peptide as defined in claim 1.

3. A fusion peptide according to claim 1 for use in treatment.

4. A fusion peptide according to claim 1, for the treatment of bacterial infection, viral infection and/or fungal infection.

5. A composition according to claim 2, for the treatment of bacterial infection, viral infection and/or fungal infection.

6. A polynucleotide encoding a peptide as defined in claim 1.

7. A method of treatment comprising administering a therapeutically effective amount of a fusion peptide according to claim 1, to a patient in need thereof.

8. A method of treatment comprising administering a therapeutically effective amount of a composition comprising a fusion peptide according to claim 2, to a patient in need thereof.

9. (canceled)

10. A composition as defined in claim 2 for use in treatment.
